(19) United States
(12) Patent Application Publication
(10) Pub. No.: US 2009/0127092 A1
(43) Pub. Date: May 21, 2009

(54) SYSTEMS AND METHODS FOR RECOVERY OF ETHANOL

(75) Inventor: DANIEL W. TEDDER, Marietta, GA (US)

Correspondence Address: TROUTMAN SANDERS LLP;BANK OF AMERICA PLAZA, 600 PEACHTREE STREET, N.E., SUITE 5200, ATLANTA, GA, 30308-2216, US

(73) Assignee: Georgia Tech Research Corporation, Atlanta, GA (US)

(21) Appl. No.: 12/268,433

(22) Filed: Nov. 10, 2008

Related U.S. Application Data
(60) Provisional application No. 60/986,317, filed on Nov. 8, 2007.
(60) Provisional application No. 60/986,325, filed on Nov. 8, 2007.

(51) Int. Cl. C07C 29/84; C07C 31/08

(52) U.S. Cl. 203/19

(57) ABSTRACT

The various embodiments of the present invention provide improved systems and methods for producing or collecting fuel grade ethanol from fermentation products. The systems and methods disclosed herein make use of extractive distillation to separate the ethanol from other components, such as water. In producing fuel grade ethanol, the systems and methods are capable of expending less than seven percent of the heating value of the ethanol.

### CROSS-REFERENCE TO RELATED APPLICATIONS

This application claims the benefit of U.S. Provisional Patent Application Nos. 60/986,317 and 60/986,325, both of which were filed 8 Nov. 2007 and are incorporated herein by reference in their entireties as if fully set forth below.

### TECHNICAL FIELD

The various embodiments of the present invention relate generally to systems and methods for producing ethanol, and more particularly, to systems and methods for producing or recovering fuel grade ethanol from fermentation products.

### BACKGROUND

Existing methods of recovering ethanol from water commonly use fractionation and adsorption techniques. For example, a beer still can be used to process a fermented product, which contains ethanol in an aqueous mixture, to produce an enriched ethanol-containing mixture that is then subjected to fractionation (e.g., fractional distillation or other like techniques). Next, the fractions containing the highest concentrations of ethanol can be passed through an adsorber to remove most, if not all, of the remaining water from the ethanol.

Unfortunately, processes such as the one just described can require significant energy expenditure in order to recover an anhydrous, or at least fuel grade, ethanol. Such processes can consume about 40 percent (%) of the heating value of the ethanol recovered. For ethanol to have an increasing role as an alternative fuel source, processes for ethanol recovery must be more energy efficient.

Accordingly, there remains a need for improved processes and systems for recovering ethanol from ethanol-containing aqueous mixtures. It is to the provision of these processes and systems that the various embodiments of the present inventions are directed.

### BRIEF SUMMARY

The various embodiments of the present invention are directed to systems and processes for the recovery of fuel grade ethanol from ethanol-containing aqueous mixtures.

A method of recovering fuel grade ethanol can include producing a fermentation product that includes ethanol, water, microorganisms and a biomass material in a fermenter; delivering at least a portion of the fermentation product to a beer still to produce a mixture that includes ethanol and water; delivering at least a portion of the mixture that includes ethanol and water to an extractive distillation column; delivering a solvent to the extractive distillation column; distilling at least a portion of the ethanol from the extractive distillation column, leaving a raffinate comprising water and the solvent; and collecting the at least the portion of the ethanol from the extractive distillation column.

The method can also include one or more of the following: recycling a raffinate from the beer still to the fermenter, removing the water from the raffinate from the extractive distillation column, recycling the water from the raffinate from the extractive distillation column to the fermenter, drying the solvent of the raffinate from the extractive distillation column, or recycling the dry solvent to the extraction distillation column.

The collected ethanol is generally a fuel grade ethanol. In some instances, the fuel grade ethanol is anhydrous ethanol. Conducting the method can consume less than seven percent of a heating value of the fuel grade ethanol that is recovered. The solvent can be selected such that it does not adversely affect, or is not toxic to, the microorganisms in the fermenter.

Another method of recovering fuel grade ethanol can include producing a fermentation product that includes ethanol, water, microorganisms and a biomass material in a fermenter; delivering at least a portion of the fermentation product to a solvent extraction column; delivering a solvent to the solvent extraction column; extracting at least a portion of the ethanol from the fermentation product to produce an extract that includes ethanol, water, and solvent; delivering at least a portion of the extract to an extractive distillation column; delivering additional solvent to the extractive distillation column; distilling at least a portion of the ethanol from the extractive distillation column, leaving a raffinate that includes water and the solvent; and collecting the at least the portion of the ethanol from the extractive distillation column.

The method can also include one or more of the following: recycling a raffinate from the solvent extraction column to the fermenter; removing the water from the raffinate from the extractive distillation column, recycling the water from the raffinate from the extractive distillation column to the fermenter, drying the solvent of the raffinate from the extractive distillation column, recycling the dry solvent to the extraction distillation column and/or to the solvent extraction column, or recycling the solvent of the raffinate from the extractive distillation column to the solvent extraction column.

Still another method of producing fuel grade ethanol can include producing a fermentation product that includes ethanol, water, microorganisms and a biomass material in a fermenter; delivering at least a portion of the fermentation product to a beer still to produce a distillate mixture that includes ethanol and water, while leaving a raffinate that includes water, ethanol, microorganisms and the biomass material; recycling at least a portion of the raffinate of the beer still that includes water, ethanol, microorganisms and the biomass material into the fermenter; delivering at least a portion of the distillate mixture that includes ethanol and water to an extractive distillation column; delivering a solvent to the extractive distillation column; distilling at least a portion of the ethanol from the extractive distillation column, leaving a raffinate that includes water and the solvent; collecting the at least the portion of the ethanol from the extractive distillation column; discharging the raffinate of the extractive distillation column from the extractive distillation column; removing at least a portion of the water from the discharged raffinate of the extractive distillation column, leaving a wet solvent; recycling at least a portion of the removed water from the discharged raffinate of the extractive distillation column into the fermenter; delivering at least a portion of the wet solvent to a carrier gas stripper; delivering a carrier gas to the carrier gas stripper; discharging a dry solvent from the carrier gas stripper, leaving a wet carrier gas; discharging at least a portion of the wet carrier gas from the carrier gas stripper; removing water from the wet carrier gas to leave a dry carrier gas; recycling at least a portion of the water removed from the wet carrier gas into the fermenter; and recycling at least a portion of the dry carrier gas into the carrier gas stripper.

Yet another method of producing fuel grade ethanol can include producing a fermentation product that includes ethanol, water, microorganisms and a biomass material in a fermenter; delivering at least a portion of the fermentation product to a solvent extraction column; delivering a solvent to the solvent extraction column; extracting at least a portion of the ethanol from the fermentation product to produce an extract that includes ethanol, water, and solvent, while leaving a raffinate that includes water, ethanol, microorganisms and the biomass material; recycling at least a portion of the raffinate of the solvent extraction column that includes water, ethanol, microorganisms and the biomass material into the fermenter; delivering at least a portion of the extract to an extractive distillation column; delivering additional solvent to the extractive distillation column; distilling at least a portion of the ethanol from the extractive distillation column, leaving a raffinate that includes water and the solvent; collecting the at least the portion of the ethanol from the extractive distillation column; discharging the raffinate of the extractive distillation column from the extractive distillation column; removing at least a portion of the water from the discharged raffinate of the extractive distillation column, leaving a wet solvent; recycling at least a portion of the removed water from the discharged raffinate of the extractive distillation column into the fermenter; delivering at least a portion of the wet solvent to a carrier gas stripper and/or to the solvent extraction column; delivering a carrier gas to the carrier gas stripper; discharging a dry solvent from the carrier gas stripper, leaving a wet carrier gas; discharging at least a portion of the wet carrier gas from the carrier gas stripper; removing water from the wet carrier gas to leave a dry carrier gas; recycling at least a portion of the water removed from the wet carrier gas into the fermenter; and recycling at least a portion of the dry carrier gas into the carrier gas stripper. The carrier gas can be one or more of nitrogen, argon, helium, carbon dioxide, or FREON.

Other aspects and features of embodiments of the present invention will become apparent to those of ordinary skill in the art, upon reviewing the following detailed description in conjunction with the accompanying figures.

### BRIEF DESCRIPTION OF THE DRAWINGS

FIG. 1 is a schematic illustration of a process for recovering fuel grade alcohol in accordance with some embodiments of the present invention.

FIG. 2 is a schematic illustration of a process for recovering fuel grade alcohol in accordance with other embodiments of the present invention.

FIG. 3 is a process flow diagram illustrating a continuous system for recovering fuel grade alcohol in accordance with some embodiments of the present invention.

FIG. 4 is a process flow diagram illustrating a portion of a continuous system for recovering fuel grade alcohol in accordance with other embodiments of the present invention.

FIG. 5 is a process flow diagram illustrating a portion of a continuous system for recovering fuel grade alcohol, that when taken with FIG. 4 illustrates the overall system, in accordance with other embodiments of the present invention.

FIG. 6 is a process flow diagram illustrating a portion of a continuous system for recovering fuel grade alcohol in accordance with other embodiments of the present invention.

FIG. 7 is a process flow diagram illustrating a portion of a continuous system for recovering fuel grade alcohol, that when taken with FIG. 6 illustrates the overall system, in accordance with other embodiments of the present invention.

### DETAILED DESCRIPTION

Referring now to the figures, wherein like reference numerals represent like parts throughout the several views, exemplary embodiments of the present invention will be described in detail. Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another. Further, the terms “a”, “an”, and “the” do not denote a limitation of quantity, but rather denote the presence of “at least one” of the referenced item.

The various embodiments of the present invention provide improved processes and systems for recovering fuel grade ethanol from ethanol-containing aqueous mixtures that are generally produced by fermentation. As used herein, “fuel grade ethanol” refers to an ethanol product that contains greater than or equal to about 96 percent (%) ethanol by volume. In some embodiments, the recovered fuel grade ethanol can be anhydrous ethanol or absolute ethanol, which contains less than about 1% non-ethanol products (e.g., water) by volume. The systems and processed described herein all make use of extractive distillation to at least partially separate ethanol from other fermentation products.

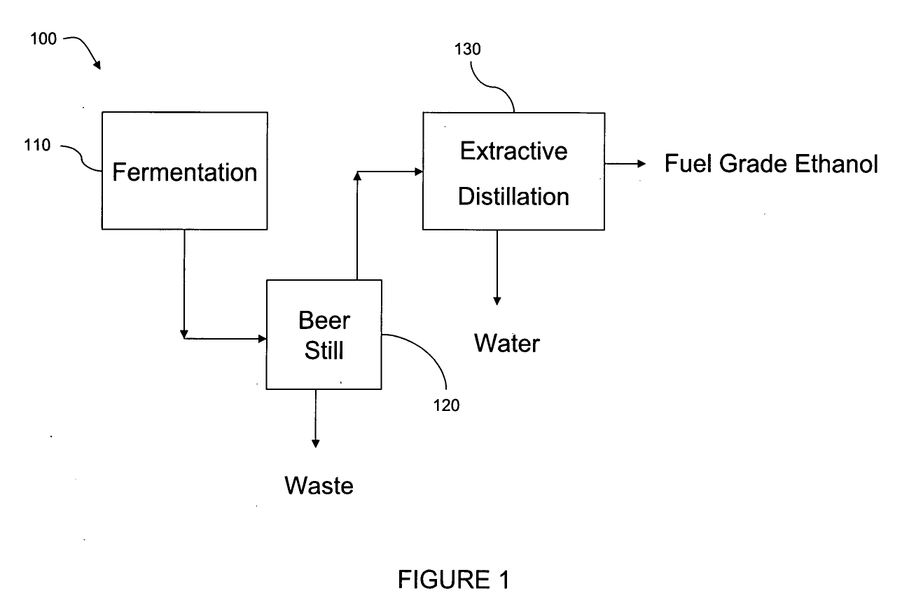

Referring now to FIG. 1, there is shown a schematic illustration of a process for recovering fuel grade ethanol, generally designated with the reference numeral 100. Although any known procedure for producing ethanol can be used, the process 100 shown in FIG. 1 begins with fermentation 110. Fermentation generally comprises culturing a microorganism (e.g., yeast, thermophilic bacterium, or the like) for a period of time in a sugar-containing medium. The basic product of the metabolism of the yeast or bacteria is alcohol. Biomass raw materials, other than sugar, can also be used in fermentation. These sources include grains (e.g., wheat, corn, barley and the like) and cellulosic materials (e.g., pine, switchgrass, wood chips, and the like). When cellulosic materials are used, however, they must undergo hydrolysis to glucose before the material can be fermented to ethanol.

The fermentation products, also termed herein as a “beer,” “broth,” or “fermentation liquor,” can include ethanol, water, microorganisms, and the sugar-containing medium. The fermentation products can be passed through a beer still 120 in which the ethanol and water are separated from both the liquor and the nonvolatile dissolved solids within the liquor. The solid-free liquid product obtained from this step is enriched in ethanol, but not yet capable of being used as a fuel.

In order to overcome the azeotrope that is formed between water and ethanol, extractive distillation 130 is used to separate the ethanol from the water. The extractive distillation step will employ a separation solvent, which is less volatile than ethanol and water, has a higher boiling point than ethanol and water, is miscible with the mixture of ethanol and water, and does not form an azeotropic mixture with either. The solvent interacts differently with the ethanol and water, causing their relative volatilities to change. This enables the new three-part mixture to be separated by normal distillation. The ethanol separates out as the top product; and the bottom product includes a mixture of the solvent and water, which optionally can be separated easily because the solvent does not form an azeotrope with water.

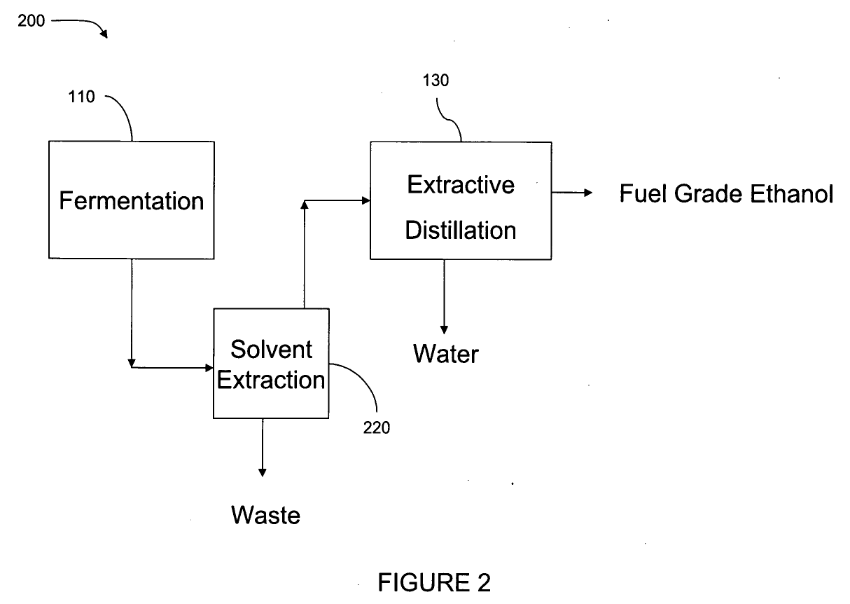

In an alternative process, shown in FIG. 2 and generally designated 200, a solvent extraction step 220 can be implemented instead of a beer still step. That is, the solvent extraction 220 can be performed after the fermenting step 110 and before the extractive distillation step 130. The solvent extraction step can be used to separate the at least a portion of the ethanol from the remainder of the fermentation products based on its solubility. The solvent extraction step will employ a solvent that, in exemplary embodiments, can also be the same solvent used for the subsequent extractive distillation step. Thus, the properties of the solvent extraction solvent should be the same as the extractive distillation solvent. A portion of the ethanol is removed from the fermentation products in the form of a solvent/ethanol/water mixture, while leaving behind a large portion of the water from the beer, residual alcohol fermentation microorganisms, and solids in the raffinate.

In exemplary embodiments, the extract (i.e., the mixture of the solvent, ethanol, and water) is heated to a vapor in preparation for the extractive distillation step.

This process 200 can be more desirable for recovering fuel grade ethanol from dilute fermentation products (i.e., where the ethanol concentration is less than or equal to about 10% of the beer). The solvent extraction step can serve to reduce the concentration of the water in the beer significantly before the extractive distillation step removes the remaining water to produce the fuel grade ethanol. The energy required to perform these two steps can be significantly lower than that required to pass the dilute beer through a beer still followed by the extractive distillation.

In some instances, the waste collected from the beer still step 120 (of process 100) or the solvent extraction step 220 (of process 200) can be recycled back into the fermenter, provided that there are no components that would have an adverse effect on the fermentation process. Similarly, as long as there are no adverse effects, the water-containing composition from the extractive distillation step 130 can be recycled back into the fermenter.

In other instances, the solvent from the solvent extraction step 220 and/or from the extractive distillation step 120 can be recycled. This can be accomplished by a stripping step. An exemplary stripping technique is carrier gas stripping. In this manner, the solvent can be reused for subsequent solvent extractions 220 and/or extractive distillations 120.

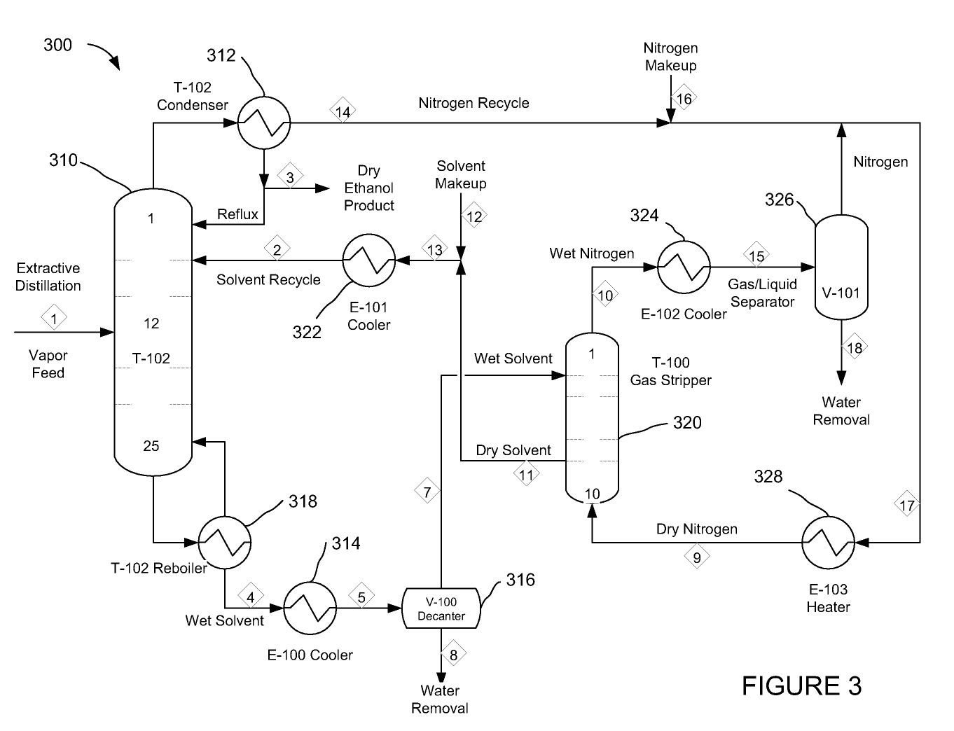

In certain exemplary embodiments, the processes and systems are capable of producing fuel grade ethanol continuously, rather than in discrete batches. Such designs can significantly reduce the energy expenditures necessary to recover the fuel grade ethanol. For example, by implementing the continuous systems shown in FIG. 3 through 7, less than about 30% of heating value of the ethanol recovered is consumed. In some embodiments, less than about 10% of heating value of the ethanol recovered is consumed. In certain situations, less than about 7% of heating value of the ethanol recovered is consumed.

The fuel grade ethanol recovery system, designated by reference numeral 300 as shown in FIG. 3, comprises an extractive distillation column 310 that is downstream of a beer still (not shown), which itself is downstream of a fermenter (not shown).

The beer still is operated as understood by those skilled in the art to produce a vapor feed comprising an ethanol-water azeotrope. The vapor feed flows from the beer still into the extractive distillation column 310. A solvent is introduced into the extractive distillation column 310 at a location higher than the entry point of the vapor feed. The temperature of the solvent should between the boiling points of water and ethanol. The temperature of the solvent should be sufficiently high to enable ethanol vapor to exit the extractive distillation column 310, but low enough to condense the water vapors and force them to settle to the bottom of the extractive distillation column 310 along with the solvent. In this manner, very little, if any, solvent is distilled out of the extractive distillation column 310. The volatility of the ethanol is much greater than that of water in the presence of the solvent, thereby enabling the extractive distillation column 310 to have very few stages compared to a conventional equivalent distillation column.

The ethanol separates out from the solvent and water vapors in the extractive distillation column 310 as the top product, meaning that it is distilled off and exits from the top of the column 310. The distilled ethanol is then processed through a condenser 312. Any gases that are noncondensible at the temperatures employed during this process 100, which may have been introduced to the extractive distillation column 310 from the ethanol-water vapor feed or from the solvent feed, also flow into the condenser 312. These noncondensible gases, which do not condense to liquid form in the condenser 312, can be collected for further use as will be described later. Additionally, if the distilled ethanol contains any other components that are not desired, it can be refluxed back to the extractive distillation column 310 from the condenser 312 for further distillation.

The bottom product (i.e., the product that exits from the bottom of the extractive distillation column 310) includes a mixture of the solvent and water, which optionally can be separated easily because the solvent does not form an azeotrope with water. The separation begins by cooling the solvent and water vapors in a cooler (i.e., heat exchanger) 314, followed by removal of at least a portion of the water in a decanter 316. If a reboiler 318 is optionally used to aid in the extractive distillation step 130, then the solvent and water vapors are passed through the reboiler 318 before they are passed through the cooler 314.

The water that is separated from the solvent can be recycled back into the fermenter, if desired. It should be noted that any solvent that has also been decanted would be introduced into the fermenter. Thus, in situations where recycling of the water is desirable, the solvent should be chosen with consideration towards its effects on the microorganisms within the fermenter and the fermentation process 110.

To recycle the solvent for subsequent use, the partially wet solvent (i.e., solvent that still contains unseparated water) is removed from the decanter 316 and is passed through a gas stripper column 320. The wet solvent is introduced to the gas stripper column 320 near the top of the column. A carrier gas (e.g., nitrogen, argon, helium, carbon dioxide, FREON, or the like) is introduced to the gas stripper column 320 near the bottom of the column. The carrier gas must be sufficiently dry and warm to cause the water to evaporate from the solvent. The evaporated residual water and carrier gas form the top product, while the dried solvent forms the bottom product from the carrier gas stripper column 320.

The dry solvent can be recycled directly back into the extractive distillation column 310. If necessary, an optional cooler 322 can be used to cool the dry solvent to a temperature suitable for use during the extractive distillation step 130. If the solvent level drops to a point where the recycled solvent is not sufficient to maintain the continuity of the process 100, additional solvent can be introduced into the system 300 upstream of the cooler 322 and downstream of the carrier gas stripper columns 320.

After being removed from the carrier gas stripper column 320, the wet (from the evaporated water) carrier gas can be passed through a cooler 324 to condense the water vapors for separation from the carrier gas. The condensed water can be separated from the carrier gas in a decanter 326. Just as with the water from decanter 316, the water collected from decanter 326 can be recycled back into the fermenter, if desired.

From the decanter 326, the now dry carrier gas can be recycled back into the carrier gas stripper column 320 for use in drying the solvent. Prior to re-entry into the carrier gas stripper column 320, the dry carrier gas will have to be heated to the appropriate temperature. This can be accomplished using a heater 328. If the carrier gas level drops to a point where the recycled carrier gas is not sufficient to maintain the continuity of the process 100, additional gas can be introduced into the system 300 upstream of the heater 328. As described above, any gases that exited the top of the extractive distillation column 310 (along with the ethanol) can also be recycled into the carrier gas stripper column 320.

As described above, and shown in FIG. 3, the system 300 provides a mechanism for producing fuel grade ethanol continuously and without significant costs associated with loss of materials. Most of the energy use in this process occurs in reboiler 318. All of the heat exchangers (i.e., heaters or coolers) have energy uses that are significantly lower than the reboiler 318. Thus, the advantages of this process are significantly reduced energy and capital costs, and the reduction or elimination of secondary waste production.

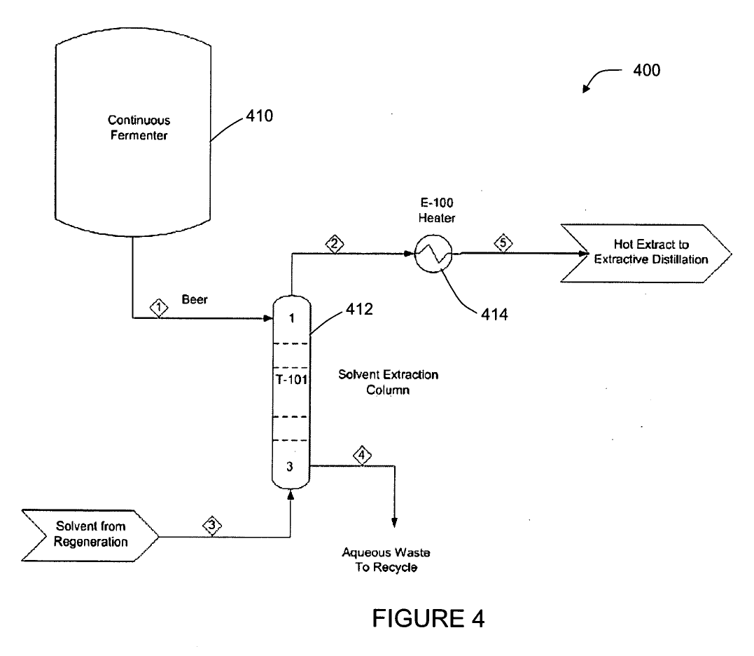

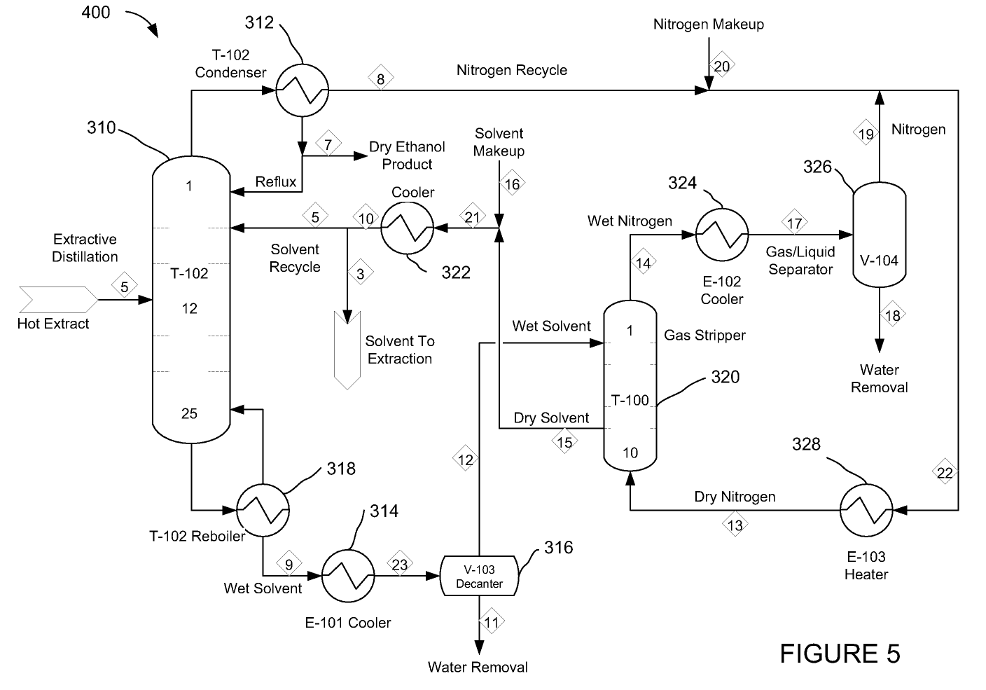

Another continuous system 400 is split into two portions, shown in FIGS. 4 and 5, for convenience. The portion of system 400 shown in FIG. 4 includes the fermentation step 110 and the solvent extraction step 220, while the portion shown in FIG. 5 includes the extractive distillation step 130 along with the various components for recycling materials that enable the system to continuously produce fuel grade ethanol.

The fuel grade ethanol recovery system 400 comprises a fermenter 410, which is operated as understood by those skilled in the art to produce a beer. The beer is introduced into a solvent extraction column 412 near the top of the column. Solvent is introduced into the solvent extraction column 412 near the bottom of the column. In the solvent extraction column 412, the beer and the solvent interact, and the solvent acts to extract the at least a portion of the ethanol from the beer. This permits efficient liquid/liquid extraction of the ethanol from the beer at reduced costs.

The raffinate or the bottoms product from the solvent extraction column 412 can be discarded as liquid waste, or can be recycled back to the fermenter 410.

The extracted ethanol, in mixture with water and the solvent, is passed through a heater 414 to produce a vapor feed for the extractive distillation column 310, which is shown in FIG. 5. Alternatively, the extracted ethanol, in mixture with water and the solvent, can be passed through a beer still (not shown) to produce the vapor feed for the extractive distillation column 310, which is shown in FIG. 5.

The remainder of system 400 as shown in FIG. 5 is identical to system 300 as shown in FIG. 3, with the exception of the solvent recycling capabilities. As indicated in FIG. 5, at least a portion of the dry solvent can be recycled from cooler 322 to the solvent extraction column 412. This dry solvent can facilitate the solvent extraction step 220 by not introducing additional water into the solvent extraction column 412.

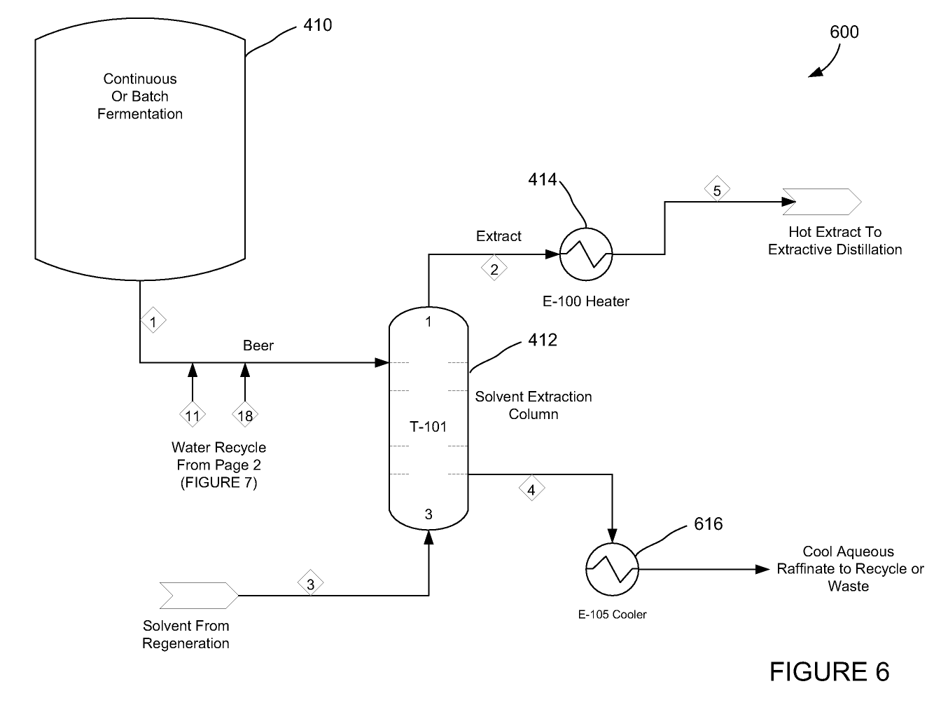

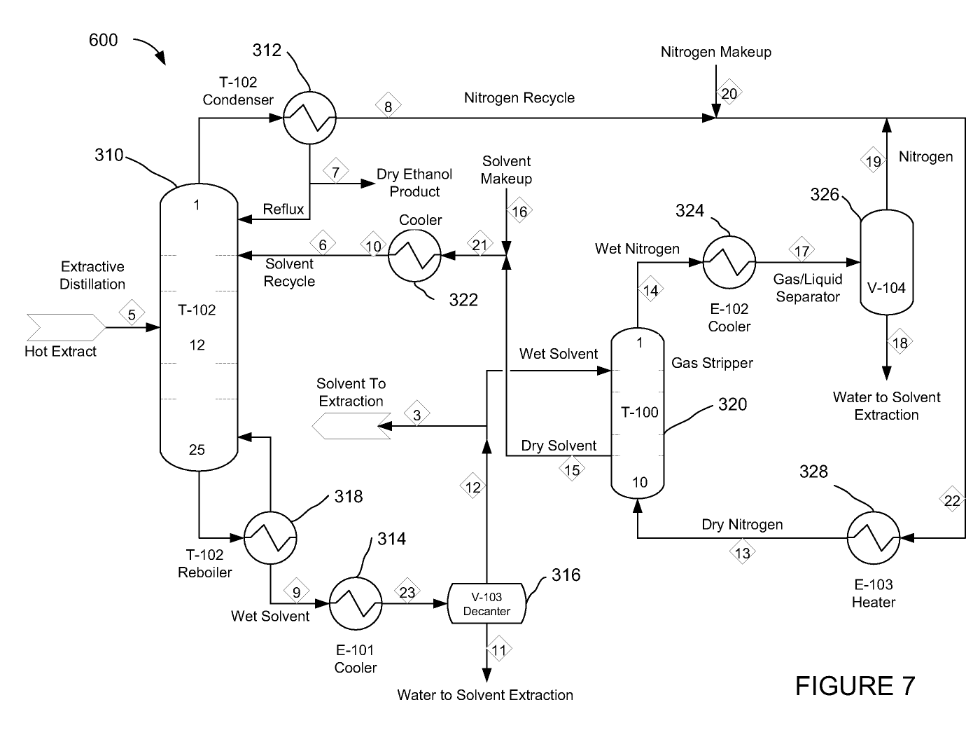

Another continuous system 600 is split into two portions, shown in FIGS. 6 and 7, for convenience. The portion of system 600 shown in FIG. 6 includes the fermentation step 110 and the solvent extraction step 220, while the portion shown in FIG. 7 includes the extractive distillation step 130 along with the various components for recycling materials that enable the system to continuously produce fuel grade ethanol.

The components of the system 600 as shown in FIG. 6 are identical to system 400 shown in FIG. 4, with the exception of the addition of cooler 616. This heat exchanger (cooler) 616 can serve to cool the raffinate from the solvent extraction column 412 before recycling it back into the fermenter 410 so that the temperature of the raffinate more closely matches the temperature of the contents of the fermenter 410.

Similarly, the components of the system 600 as shown in FIG. 7 are identical to system 400 shown in FIG. 5 (and system 300 shown in FIG. 3), with the exception of the solvent and aqueous waste recycling capabilities. As indicated in FIG. 7, at least a portion of the water (and other separated components) can be recycled from decanter 316 to the solvent extraction column 412. And, at least a portion of water can be recycled from decanter 326 to the solvent extraction column 412. In addition, instead of drying the solvent before recycling it back into the solvent extraction column 412 as shown in FIG. 5, at least a portion of the solvent can be recycled back into the solvent extraction column 412 in wet form (i.e., including some water that was not removed by decanter 316). In this manner, the energy required to dry the solvent is not expended if it is going to be exposed to water in the solvent extraction column 412.

As would be expected by using a combination of features from systems 400 and 600, a combination of dry and wet solvent can be recycled into the solvent extraction column 412. The choice of wet solvent or dry solvent for recycling back into the solvent extraction column 412 will depend on the particular system and the energy efficiency desired.

Thus, the systems 400 and 600 shown in FIGS. 4 and 5 and FIGS. 6 and 7, respectively, provide other mechanisms for producing fuel grade ethanol continuously and without significant costs associated with loss of materials. Just as with system 300, most of the energy use in systems 400 and 600 occurs in reboiler 318. As a result, this process 200 also provides significantly reduced energy and capital costs, and reduces or eliminates secondary waste production.

In terms of equipment, standard equipment items may be used for the solvent extraction column 412, extractive distillation column 310, and the carrier gas stripper column 326. Standard equipment can also be used for the fermenter 410 and beer still (not shown). This technology is well developed and a number of equipment items may be used effectively. It is preferred, however, to use a solvent extraction column 412, which is relatively gentle on the microorganisms, which can pass through it. Specifically, it would be undesirable to use a high-speed centrifugal contactor to achieve the solvent extraction step 220. Exemplary solvent extraction units 412 include a rotating disc column or a reciprocating plate column.

Pressure ranges throughout the system vary anywhere from about 100 millimeters (mm) mercury (Hg) to about 760 mm Hg. Solvent regeneration is best accomplished at about 100 mm Hg or more in order to use cooling water to condense the product rather than refrigeration.

Any solvent that meets the criteria for extractive distillation and/or solvent extraction can be used. That is, water must be less volatile than ethanol in the presence of the selected solvent, and the solvent should be less volatile than either water or ethanol in ternary mixtures of these three species. The solvent must not form stable emulsions when mixed vigorously with fermentation broths. The solvent should be chemically stable in the presence of fermentation broths or, if it undergoes degradation, the degradation products must exhibit similar properties as the solvent. The solvent should be a liquid at all operating temperatures in the process. The solvent must not be highly flammable or result in explosive conditions within the process. The solvent viscosity, density, and liquid/liquid interfacial tension with fermentation broths must be such as to enable a primary break time of less than ten minutes. If the solvent includes nonpolar diluents, then extracts should not form third phases. Determination of such solvents is well within the scope of those skilled in the art to which this disclosure pertains.

In systems and processes where continuity and recycling of the solvent are desired, then an additional requirement is placed on the solvent, namely that it must not be toxic to fermentation microorganisms. Generally, solvents that are toxic to fermentation microorganisms also have high aqueous solubilities (i.e., solubility in water). For example, if an alcohol is used as the solvent, then the alcohol must have an aqueous solubility less than that of 2-ethyl hexanol or 1-octanol. When the aqueous solubility of a solvent is small, the solvent does not interfere with microbe functions in any way.

Examples of suitable solvents for these purposes include branched chain paraffins, long chain alcohols, fatty alcohols, fatty acids, and the like. Exemplary paraffins have C₁₂ to C₁₆ backbones, such as those commercially available from EXXON under the name ISOPAR. Exemplary long chain alcohols include decanol, dodecanol, tridecanol, and longer chained (i.e., longer than C₁₀) primary, secondary, and tertiary alcohols. Exemplary fatty alcohols include (in addition to decanol, dodecanol, tridecanol) lauryl alcohol, cetearyl alcohol, cetyl alcohol, stearyl alcohol, and tallow type alcohols. Exemplary fatty acid solvents include coconut, tallow, and linseed fractions. In general, these alcohols and acids are relatively inexpensive.

In exemplary embodiments, the solvent is a C₁₀ or longer alcohol (e.g., decanol, dodecanol, and tridecanol).

The appropriate ratio of solvent to ethanol is approximately 1 to 1. However, solvent to ethanol ratios as high as 10 to 1 or a low as 1 to 10 may also be preferred for certain applications.

In an advantageous feature of some embodiments of the present invention, the coupling of solvent extraction with continuous fermentation offers the possibility of higher alcohol production rates with reduced costs. The solvent extraction is enhanced substantially by the presence of sugar in the fermentation effluent. It would be desirable to operate the fermenter at the highest possible sugar concentration, although there are some problems with this mode of operation since high sugar concentrations can also inhibit the fermentation process. Maintaining the sugar concentration in the fermenter at approximately 3% by weight results in a product having a sufficiently high sugar concentration in the effluent. The pH range is not that critical except that the fermentation should be carried out in a slightly acidic environment in order to prevent the formation of stable emulsions.

Another advantageous feature of some embodiments of the present invention is that if a solvent extraction column (and especially a reciprocating plate column) is used, the need to remove the biomass from the fermenter product is eliminated.

In embodiments where carbon dioxide is used as the carrier gas, another advantage lies in that no additional expenditure is necessary for purchasing the carrier gas. Since carbon dioxide is a byproduct or waste product of the fermentation process, it simply can be collected and used as the carrier gas.

The various embodiments of the present invention are further illustrated by the following non-limiting examples.

### EXAMPLES

All cases were modeled using the AspenTech Hysys simulator. Given feed and design input information, the simulator computes the steady state energy and material balance relationships to estimate the flow rates and compositions of internal process streams and product streams.

In the present cases, thermodynamic behavior was estimated using the NRTL model with UNIFAC interaction parameters to predict activity coefficients in the liquid phase. The UNIFAC LLE Activity Model Interaction parameters were used as estimated by Hysys except for the water and 1-decanol interaction parameter, which was adjusted slightly to a value of 100 to increase water extraction into the solvent and thereby improve fit with experimental extraction measurements. These interaction parameters and thermodynamic models are well known and can be found, for example, in J. M. Prausnitz, R. N. Lichtenthaler and C. Gomes de Azevedo, Molecular Thermodynamics of Fluid-Phase Equilibria, 3^{rd} Edition, Prentice Hall PTR, New Jersey (1999).

The extractive distillation and carrier gas stripping columns were modeled as towers in Hysys, but the solvent extraction column was modeled as three countercurrent stages of three phase (VLL) flash due to numerical instabilities. The extraction factor for each stage was about 1.3 on a mass basis. The extractive distillation column was modeled using 25 theoretical stages. The carrier gas stripper was modeled as 10 theoretical stages.

The feed was either an equimolar vapor mixture of ethanol and water as generated by a beer still, or a 15% by mass solution of ethanol in water. In the latter case, the beer was extracted using three stages of liquid/liquid extraction to produce a raffinate that is about 3% by mass ethanol. Three stages of liquid/liquid extraction under these conditions were thus estimated to provide about 84% recovery of the ethanol, but higher recoveries can be achieved with more stages as long as the extraction factor is greater than unity.

### Example 1

### Continuous Recovery of Fuel Grade Ethanol by Extractive Distillation and Carrier Gas Stripping

In this example, fuel grade ethanol recovery was simulated using the system as shown in FIG. 3. The modeled process proceeds as described below. The various diamonds with numbers contained in FIG. 3 are the stream numbers to which the description refers. Table 1 provides the data for the calculations, and is divided into five sub-parts for reading convenience.

A beer still was operated according to common practice to produce Vapor Feed 1, which comprised and ethanol and water mixture (typically about 50 mole percent ethanol). Vapor Feed 1 was passed into Extractive Distillation Column T-102. Solvent Recycle 2 was passed into T-102 near the top stage after its temperature was cooled to an appropriate temperature. This temperature must be high enough to enable ethanol vapor to pass into the Condenser T-102, but low enough to condense water vapors and force them into the bottom of Column T-102 and out with solvent into Stream 4 where they are cooled in E-100 and decanted in V 100. By this means, a portion of the water entering T-102 was removed without evaporating the water and thereby reducing energy requirements. T-102 needed about 25 theoretical stages with the vapor feed introduced about stage 12, the solvent added onto the top stage 1, and the bottoms product withdrawn at stage 25.

The Reflux Ratio in T-102 was set to a value of 1.0. The primary energy sink in this process was the 1-102 Reboiler, which, under these conditions, requires about 4.49E+5 kilojoules per hour (kJ/h) to produce about 231.8 kilograms per hour (kg/h) of ethanol product. With an ethanol heating value of 29.8 megajoules per kilogram (MJ/kg), the energy use was about 1.937E+3 kJ/kg of product or about 6.59% of the product's high heating value.

Noncondensible gases entering with either Stream 1 or Stream 2 passed through partial condenser T-102 and report to Stream 14 where they were recycled to the Gas Stripper column or discarded as waste. Makeup Carrier Gas could be added as Stream 16.

Dry ethanol product was recovered from the top of Extractive Distillation Column T-102 as Stream 3.

Wet Solvent from V-100 Decanter was removed as Stream 7 and passed into Gas Stripper Column T-100 near the top of the column. Warm dry nitrogen was passed into the bottom of T-100 as Stream 9. Stream 9 must be sufficiently warmed as to enable water evaporation from the solvent Stream 7 to produce Dry Solvent Stream 11 for recycle to T-102.

Wet vapors from T-100 were removed as Stream 10, which was cooled in E-102 to condense water in Stream 15 and enable its removal by decantation in V-101. Water was removed from V-101 as Stream 18. Dry nitrogen was recycled as Stream 17 to Heater E-103 where the gas was heated to the design temperature as required for T-100 operation. T-100 required about 10 theoretical stages as calculated.

Most of the energy use in this process occurred in Reboiler T-102. All other coolers and heaters had duties that were one or two orders of magnitude smaller than that required by Reboiler T-102. Typical conditions for operation were described in Table 1.

The process calculated described an isobaric case without indicating pumps or blowers for simplicity. Liquid pumps will be required, for example, to supply reflux to column T-102 and also to introduce Solvent Stream 6 into the top of the column. A liquid pump is also required to transfer Wet Solvent Stream 12 to the top of T-100 and a blower is required to introduce Dry Gas Stream 13 into the bottom of T-100 and to recirculate the gas (as used in the calculations, nitrogen) as shown.

For comparison, a conventional fractionator was modeled to separate water and ethanol. The same feed stream as Stream 1 was modeled for the fractionator comparison. This stream was an equimolar mixture of water and ethanol as saturated vapor at 1 atmosphere. The fractionator was modeled as a column with 100 theoretical trays (as compared to the 25 used for the extractive distillation calculation), with a reflux ratio of 30 (as compared to a reflux ratio of 1 used for the extractive distillation calculation).

TABLE 1

| | Stream Number | | | | |
|---|---|---|---|---|---|
| | 1 | 2 | 3 | 4 | 5 |
| | Stream Name | | | | |
| | | | Ethanol | | Cold |
| | Vapor Feed | Solvent | Product | T-102 Bottoms | Bottoms |
| Vapor Fraction | 1 | 0.0002 | 0 | 0.1977 | 0 |
| Temperature (C.) | 84.29 | 23 | 52.36 | 104.9 | 85 |
| Pressure (kPa) | 101.3 | 101.3 | 101.3 | 101.3 | 101.3 |
| Molar Flow (kg mole/h) | 10 | 10.2 | 5.06 | 15.12 | 15.12 |
| Mass Flow (kg/h) | 320.4 | 1599 | 231.8 | 1687 | 1687 |
| Std Ideal Liquid | 0.3796 | 1.922 | 0.2907 | 2.01 | 2.01 |
| Volume Flow (m³/h) | | | | | |
| Heat Flow (kJ/h) | −2.35E+06 | −4.76E+06 | −1.39E+06 | −5.72E+06 | −5.92E+06 |
| Molar Enthalpy (kJ/kg | −2.35E+05 | −4.67E+05 | −2.74E+05 | −3.78E+05 | −3.92E+05 |
| mole) | | | | | |
| Master Composition | 0.5 | 0.0007 | 0.9816 | 0.0022 | 0.0022 |
| Mole Fraction | | | | | |
| (Ethanol) | | | | | |
| Master Composition | 0.5 | 0.0087 | 0.0166 | 0.331 | 0.331 |
| Mole Fraction (H₂0) | | | | | |
| Master Comp Mole | 0 | 0.0013 | 0 | 0 | 0 |
| Fraction (Nitrogen) | | | | | |
| Master Composition | 0 | 0.9893 | 0.0018 | 0.6668 | 0.6668 |
| Mole Fraction (1- | | | | | |
| Decanol) | | | | | |

| | Stream Number | | | | |
|---|---|---|---|---|---|
| | 6 | 7 | 8 | 9 | 10 |
| | Stream Name | | | | |
| | V-100 Vapor | V-100 Light | V.100 Water | Nitrogen | T-100 Vapor |
| Vapor Fraction | 1 | 0 | 0 | 1 | 1 |
| Temperature (C.) | 85 | 85 | 85 | 100 | 65.31 |
| Pressure (kPa) | 101.3 | 101.3 | 101.3 | 101.3 | 101.3 |
| Molar Flow (kg mole/h) | 0 | 13.01 | 2.115 | 20.3 | 23.12 |
| Mass Flow (kg/h) | 0 | 1649 | 38.19 | 567.5 | 620.9 |
| Std Ideal Liquid Volume | 0 | 1.972 | 3.83E−02 | 0.7032 | 0.7574 |
| Flow (m³/h) | | | | | |
| Heat Flow (kJ/h) | 0 | −5.33E+06 | −5.93E+05 | 1.14E+04 | −6.91E+05 |
| Molar Enthalpy (kJ/kg | −2.40E+05 | −4.1E+−5 | −2.80E+05 | 560.9 | −2.99E+04 |
| mole) | | | | | |
| Master Composition | 0.0073 | 0.0025 | 0.0005 | 0.0004 | 0.0013 |
| Mole Fraction (Ethanol) | | | | | |
| Master Composition | 0.989 | 0.2223 | 0.9993 | 0.0064 | 0.1268 |
| Mole Fraction (H₂0) | | | | | |
| Master Comp Mole | 0 | 0 | 0 | 0.9932 | 0.8713 |
| Fraction (Nitrogen) | | | | | |
| Master Composition | 0.0037 | 0.7752 | 0.0002 | 0 | 0.0006 |
| Mole Fraction | | | | | |
| (1-Decanol) | | | | | |

| | Stream Number | | | | |
|---|---|---|---|---|---|
| | 11 | 12 | 13 | 14 | 15 |
| | Stream Name | | | | |
| | T-100 | Solvent | | | |
| | Recycle | Makeup | Hot Solvent | T-102 Vapor | Cool N₂ |
| Vapor Fraction | 0 | 0 | 0 | 1 | 0.8776 |
| Temperature (C.) | 59.17 | 23 | 59.11 | 52.36 | 1 |
| Pressure (kPa) | 101.3 | 101.3 | 101.3 | 101.3 | 101.3 |
| Molar Flow (kg | 10.18 | 1.81E−02 | 10.2 | 2.00E−02 | 23.12 |
| mole/h) | | | | | |
| Mass Flow (kg/h) | 1596 | 2.87 | 1599 | 0.6749 | 620.9 |
| Std Ideal Liquid | 1.918 | 3.45E−03 | 1.921 | 8.41E−04 | 0.7574 |
| Volume Flow (m³/h) | | | | | |
| Heat Flow (kJ/h) | −4.63E+06 | −8505 | −4.63E+06 | −1511 | −8.63E+05 |
| Molar Enthalpy | −4.54E+05 | −4.69E+05 | −4.54E+05 | −7.55E+04 | −3.73E+04 |
| (kJ/kg mole) | | | | | |
| Master Composition | 0.001 | 0 | 0.001 | 0.3205 | 0.0013 |
| Mole Fraction | | | | | |
| (Ethanol) | | | | | |
| Master Composition | 0.010 | 0 | 0.0091 | 0.0056 | 0.1268 |
| Mole Fraction (H₂0) | | | | | |
| Master Comp Mole | 0.0013 | 0 | 0.0013 | 0.6739 | 0.8713 |
| Fraction (Nitrogen) | | | | | |
| Master Composition | 0.9886 | 1 | 0.9886 | 0 | 0.0006 |
| Mole Fraction (1- | | | | | |
| Decanol) | | | | | |

| | Stream Number | | | | |
|---|---|---|---|---|---|
| | 16 | 17 | 18 | | |
| | Stream Name | | | | |
| | N2 Makeup | N2 Recycle | Water Recycle | E-100 Duty | E-101 Duty |
| Vapor Fraction | 1 | 1 | 0 | | |
| Temperature (C.) | 23 | 1.057 | 1 | | |
| Pressure (kPa) | 101.3 | 101.3 | 101.3 | | |
| Molar Flow (kg | −1.35E−02 | 20.3 | 2.83 | | |
| mole/h) | | | | | |
| Mass Flow (kg/h) | −0.3771 | 567.7 | 53.5 | | |
| Std Ideal Liquid | −4.68E−04 | 0.7035 | 5.43E−02 | | |
| Volume Flow (m³/h) | | | | | |
| Heat Flow (kJ/h) | 0.7844 | −5.07E+04 | −8.14E+05 | 2.05E+05 | 1.26E+05 |
| Molar Enthalpy | −58.27 | −2499 | −2.88E+05 | | |
| (kJ/kg mole) | | | | | |
| Master Composition | 0 | 0.001 | 0.0053 | | |
| Mole Fraction | | | | | |
| (Ethanol) | | | | | |
| Master Composition | 0 | 0.0065 | 0.9894 | | |
| Mole Fraction (H₂0) | | | | | |
| Master Comp Mole | 1 | 0.9925 | 0 | | |
| Fraction (Nitrogen) | | | | | |
| Master Composition | 0 | 0 | 0.0053 | | |
| Mole Fraction (1- | | | | | |
| Decanol) | | | | | |

| | Stream Name | | | |
|---|---|---|---|---|
| | | | | T-102 Reboiler |
| | E-102 Duty | E-103 Duty | T-102 Duty | duty |
| Heat Flow (kJ/h) | 1.72E+05 | 5.90E+04 | 4.43E+05 | 4.49E+05 |

The distillate was taken at about the same molar rate as in the extractive distillation column, but it was only 92.5% ethanol as opposed to about 98.5% in extractive distillation, as seen in Table 1. Also, the bottoms product from the fractionator contains about 6.5% ethanol whereas the bottoms from the extractive distillation column are less than 1% ethanol.

The energy requirements are estimated as 2.646E+04 kJ/kg for 92.5% ethanol distillate. With the high heating value of ethanol taken as 29.8 MJ/kg, this duty was about 88.7% of the heating value of the product. In other words, the extractive distillation process is much smaller than a conventional fractionator and the energy use was about an order of magnitude less than for fractionation.

Equally important, the extractive distillation process made the product even dryer than 98.5%, but the fractionator could not go above the azeotrope (about 95% ethanol). Also, the ethanol recovery in extractive distillation was higher than would be for a fractionator.

All of these features clearly demonstrate the superiority of extractive distillation over conventional fractionation for this separation.

### Example 2

### Continuous Recovery of Fuel Grade Ethanol by Solvent Extraction, Extractive Distillation, and Carrier Gas Stripping

In this example, fuel grade ethanol recovery was simulated using the system as shown in FIGS. 4 and 5. The modeled process proceeds as described below. The various diamonds with numbers contained in FIGS. 4 and 5 are the stream numbers to which the description refers. Table 2 provides the data for the calculations, and is divided into five sub-parts for reading convenience.

The process was isobaric. For simplicity, pumps and blowers were been included in FIGS. 4 and 5. Liquid metering pumps were required to control Streams 1 and 2. A liquid pump could be required to transfer Hot Extract Stream 5 into Extractive Distillation Column T-102. T-102 required a reflux pump and a pump to transport Stream 6 into the top of T-102. A liquid pump was required to transport Stream 12 into the top of T-100. In addition, a blower was required to recirculate the carrier gas Stream 13 through T-100.

As depicted, Streams 1 and Solvent Stream 3 were pumped counter currently into a suitable liquid/liquid contactor. The Raffinate Stream 4 can be discarded as liquid waste or recycled back to the continuous fermenter depending upon the application. Extract Stream 2 was passed through a heater E-100 to produce Stream 5, which was pumped midway into T-102. Additional solvent Stream 6 was pumped to the top of T-102 to provide solvent to dry the ethanol product. Fuel grade ethanol was recovered as Stream 7 on Page 2.

Hot solvent and water were discharged as Wet Solvent Stream 9 from the bottom of T-102. Stream 9 may be cooled to produce two liquid phases (water and solvent). The water may be removed from Decanter V-103 as Stream 11. Extract from V-103 was pumped into the top of dryer column T-100 to evaporate water. Warm dry nitrogen (as the carrier gas) was blown into the bottom of Gas Stripper T-100 to evaporate residual water and produce Dry Solvent Stream 15, which was cooled in E-104 and adjusted to the correct temperature for use in T-101 and T-102 (Streams 3 and 6, respectively).

As, with the system in EXAMPLE 1, the T-102 Reboiler was the largest energy sink in this process. With a Reflux Ratio of 1.0, the estimated duty was about 1.11E+6 kJ/h to produce 241.3 kg/h of 98+% ethanol product. Assuming an ethanol heating value of 29.8 MJ/kg, the energy use in the T-102 Reboiler was about 4600 kJ/kg or about 15.4% of the product heating value. All other duties were smaller than T-102 and some could have been reduced by optional heat exchanger matching (not shown in the Figures).

Wet Nitrogen Stream 14 was taken from the top of column T-100 and passed through Cooler E-102, where water was condensed and separated as Stream 18 in V-104. Stream 19 was mixed with Stream 8 and Stream 20 as needed to produce recycle Stream 22, which was heated to the correct temperature in E-103 for use in T-100.

The use of V-103 to remove water reduced the need to evaporate water in T-100 and reduced energy use. Additional improvements could have been gained by splitting Stream 12, which is described in EXAMPLE 3 below.

The theoretical stage requirements were modest. Extraction column T-101 was modeled as three counter current stages, but could have been improved by using as many as 20 theoretical stages to maximize the ethanol recovery from the beer. Extractive distillation column T-102 required about 25 theoretical stages with the feed introduced around stage 12. The Gas Stripper, Column T-100, required about 10 stages.

TABLE 2

| | Stream Number | | | | |
|---|---|---|---|---|---|
| | 1 | 2 | 3 | 4 | 5 |
| | Stream Name | | | | |
| | Beer | Extract | Solvent | Raffinate | Hot Extract |
| Vapor Fraction | 0 | 0 | 0.0002 | 0 | 0.313 |
| Temperature (C.) | 23 | 23 | 23 | 23 | 100 |
| Pressure (kPa) | 101.3 | 101.3 | 101.3 | 101.3 | 101.3 |
| Molar Flow (kg mole/h) | 100 | 25.03 | 12.7 | 87.53 | 25.03 |
| Mass Flow (kg/h) | 1983 | 2353 | 1982 | 1607 | 2353 |
| Standard Ideal Liquid | 2.062 | 2.816 | 2.383 | 1.622 | 2.816 |
| Volume Flow (m³/h) | | | | | |
| Heat Flow (kJ/h) | −2.85E+07 | −9.39E+06 | −5.91E+06 | −2.49E+07 | −8.63E+06 |
| Molar Enthalpy (kJ/kg | −2.85E+05 | −3.75E+05 | −4.66E+05 | −2.85E+05 | −3.45E+05 |
| mole) | | | | | |
| Master Composition Mole | 0.0646 | 0.2156 | 0.0062 | 0.0118 | 0.2156 |
| Fraction (Ethanol) | | | | | |
| Master Composition Mole | 0.9354 | 0.2856 | 0.0099 | 0.9881 | 0.2856 |
| Fraction (H₂O) | | | | | |
| Master Composition Mole | 0 | 0.4986 | 0.9826 | 0 | 0.4986 |
| Fraction (1-Decanol) | | | | | |
| Master Composition Mole | 0 | 0 | 0 | 0 | 0 |
| Fraction (N₂) | | | | | |
| | Stream Number | | | | |
| | 6 | 7 | 8 | 9 | 10 |
| | Stream Name | | | | |
| | | Ethanol | T-102 NC | T-102 | Cold Solvent |
| | Solvent 2 | Product | Gases | Bottoms | Recycle |
| Vapor Fraction | 0.0002 | 0 | 1 | 0.1701 | 0.0002 |
| Temperature (C.) | 23 | 23 | 23 | 119.4 | 23 |
| Pressure (kPa) | 101.3 | 101.3 | 101.3 | 101.3 | 101.3 |
| Molar Flow (kg mole/h) | 10.4 | 5.26 | 2.16E−02 | 30.14 | 23.1 |
| Mass Flow (kg/h) | 1622 | 241.3 | 0.6311 | 3733 | 3605 |
| Standard Ideal Liquid | 1.949 | 0.3027 | 7.84E−04 | 4.462 | 4.333 |
| Volume Flow (m³/h) | | | | | |
| Heat Flow (kJ/h) | −4.84E+06 | −1.47E+06 | −358.1 | −1.17E+07 | −1.08E+07 |
| Molar Enthalpy (kJ/kg | −4.66E+05 | −2.79E+05 | −1.66E+04 | −3.88E+05 | −4.66E+05 |
| mole) | | | | | |
| Master Composition Mole | 0.0062 | 0.9845 | 0.0673 | 0.0093 | 0.0062 |
| Fraction (Ethanol) | | | | | |
| Master Composition Mole | 0.0099 | 0.0138 | 0.001 | 0.2381 | 0.0096 |
| Fraction (H₂O) | | | | | |
| Master Composition Mole | 0.9826 | 0.0017 | 0 | 0.7525 | 0.9829 |
| Fraction (1-Decanol) | | | | | |
| Master Composition Mole | 0.0013 | 0 | 0.9317 | 1E−04 | 0.0013 |
| Fraction (N₂) | | | | | |

| | Stream Number | | | | |
|---|---|---|---|---|---|
| | 11 | 12 | 13 | 14 | 15 |
| | Stream Name | | | | |
| | | V-103 | Dry | | T-100 Dry |
| | V-103 Water | Solvent | Nitrogen | T-100 Water | Solvent |
| Vapor Fraction | 0 | 0 | 1 | 1 | 0 |
| Temperature (C.) | 85 | 85 | 100 | 65.15 | 58.49 |
| Pressure (kPa) | 101.3 | 101.3 | 101.3 | 101.3 | 101.3 |
| Molar Flow (kg mole/h) | 0.5906 | 29.55 | 45.1 | 51.61 | 23.05 |
| Mass Flow (kg/h) | 10.69 | 3722 | 1263 | 1388 | 3597 |
| Standard Ideal Liquid | 1.07E−02 | 4.451 | 1.565 | 1.693 | 4.323 |
| Volume Flow (m³/h) | | | | | |
| Heat Flow (kJ/h) | −1.66E+05 | −1.21E+07 | 2864 | −1.61E+06 | −1.05E+07 |
| Molar Enthalpy (kJ/kg | −2.80E+05 | −4.08E+05 | 63.51 | −3.13E+04 | −4.53E+05 |
| mole) | | | | | |
| Master Composition Mole | 0.002 | 0.0095 | 0.0026 | 0.0049 | 0.0062 |
| Fraction (Ethanol) | | | | | |
| Master Composition Mole | 0.9978 | 0.223 | 0.0064 | 0.129 | 0.0097 |
| Fraction (H₂O) | | | | | |
| Master Composition Mole | 0.0002 | 0.7676 | 0 | 0.0006 | 0.9829 |
| Fraction (1-Decanol) | | | | | |
| Master Composition Mole | 0 | 0 | 0.991 | 0.8655 | 0.0012 |
| Fraction (N₂) | | | | | |

| | Stream Number | | | | |
|---|---|---|---|---|---|
| | 16 | 17 | 18 | 19 | 20 |
| | Stream Name | | | | |
| | Solvent | | | Cold N₂ | |
| | Makeup | Cool N₂ | V-104 Water | Recycle | N₂ Makeup |
| Vapor Fraction | 0 | 0.8733 | 0 | 1 | 1 |
| Temperature (C.) | 23 | 1 | 1 | 1 | 23 |
| Pressure (kPa) | 101.3 | 101.3 | 101.3 | 101.3 | 101.3 |
| Molar Flow (kg mole/h) | 5.35E−02 | 51.61 | 6.54 | 45.07 | 1.17E−02 |
| Mass Flow (kg/h) | 8.466 | 1388 | 126.3 | 1262 | 0.3287 |
| Standard Ideal Liquid | 1.02E−02 | 1.693 | 0.1292 | 1.563 | 4.08E−04 |
| Volume Flow (m³/h) | | | | | |
| Heat Flow (kJ/h) | −2.51E+04 | −2.01E+06 | −1.88E+06 | −1.28E+05 | −0.6837 |
| Molar Enthalpy (kJ/kg | −4.69E+05 | −3.89E+04 | −2.88E+05 | −2834 | −58.27 |
| mole) | | | | | |
| Master Composition Mole | 0 | 0.0049 | 0.0211 | 0.0026 | 0 |
| Fraction (Ethanol) | | | | | |
| Master Composition Mole | 0 | 0.129 | 0.9738 | 0.0064 | 0 |
| Fraction (H₂O) | | | | | |
| Master Composition Mole | 1 | 0.0006 | 0.005 | 0 | 0 |
| Fraction (1-Decanol) | | | | | |
| Master Composition Mole | 0 | 0.8655 | 1E−04 | 0.991 | 1 |
| Fraction (N₂) | | | | | |

| | Stream Number | | |
|---|---|---|---|
| | 21 | 22 | 23 |
| | Stream Name | | |
| | Hot Solvent | | T-102 Cold |
| | Recycle | N₂ Recycle | Bottoms |
| Vapor Fraction | 0 | 1 | 0 |
| Temperature (C.) | 58.41 | 1.017 | 85 |
| Pressure (kPa) | 101.3 | 101.3 | 101.3 |
| Molar Flow (kg mole/h) | 23.1 | 45.1 | 30.14 |
| Mass Flow (kg/h) | 3605 | 1263 | 3733 |
| Standard Ideal Liquid | 4.333 | 1.565 | 4.462 |
| Volume Flow (m³/h) | | | |
| Heat Flow (kJ/h) | −1.05E+07 | −1.28E+05 | −1.22E+07 |
| Molar Enthalpy (kJ/kg | −4.53E+05 | −2840 | −4.06E+05 |
| mole) | | | |
| Master Composition Mole | 0.0062 | 0.0026 | 0.0093 |
| Fraction (Ethanol) | | | |
| Master Composition Mole | 0.0096 | 0.0064 | 0.2381 |
| Fraction (H₂O) | | | |
| Master Composition Mole | 0.9829 | 0 | 0.7525 |
| Fraction (1-Decanol) | | | |
| Master Composition Mole | 0.0013 | 0.991 | 1E−04 |
| Fraction (N₂) | | | |

| | Stream Name | | | | | | |
|---|---|---|---|---|---|---|---|
| | | | | | | T-102 | T-102 |
| | E-100 | E-101 | E-102 | E-103 | E-104 | Cond | Reboiler |
| | Duty | Duty | Duty | Duty | Duty | Duty | duty |
| Heat Flow (kJ/h) | 7.60E+05 | 5.25E+05 | 3.95E+05 | 1.31E+05 | 2.80E+05 | 5.08E+05 | 1.11E+06 |

### Example 3

### Continuous Recovery of Fuel Grade Ethanol by Solvent Extraction, Extractive Distillation, and Carrier Gas Stripping

In this example, fuel grade ethanol recovery was simulated using the system as shown in FIGS. 6 and 7. The modeled process proceeded as described in EXAMPLE 2, with the exception that Stream 12 was split into two streams with partial recycle of Stream 12 directly back to T-101 as Stream 3 without drying.

### Example 4

### Continuous Recovery of Fuel Grade Ethanol by Solvent Extraction, Extractive Distillation, and Carrier Gas Stripping

In this example, the processes described in EXAMPLES 1-3 were carried out using the feed rate, feed purity, extractive distillation rate, number of trays for extractive distillation, and condenser pressure parameters shown in Table 3. The results of these calculations (i.e., reboiler duty, extractive distillation distillate content, and ethanol recovery), are also provided in Table 3. Furthermore, Table 4 provides comparative data using conventional fractionation processes.

TABLE 3
Data Simulated According to Processes in EXAMPLES 1-3

| | | | | | | | Reboiler | | | |
|---|---|---|---|---|---|---|---|---|---|---|
| | Ethanol | | T-102 | | | T-102 | Duty, | Reboiler | | T-102 |
| | Feed | Feed | Rate | Distillate | Ethanol | Reboiler | (MJ/kg | Duty | | Condenser |
| | Rate | Purity | (kg- | (mole % | Recovery | Duty | Ethanol | (% Ethanol | T-102 | Pressure |
| Case | kg/h | (mole %) | mole/h) | Ethanol) | (mole %) | (MJ/h) | Recovered) | HHV) | Trays | (kPa) |
| 1 | 230.00 | 50.00 | 5.06 | 98.16 | 99.33 | 448.50 | 1.96 | 6.59 | 25 | 101.4 |
| 2 | 296.94 | 6.46 | 6.40 | 98.51 | 97.68 | 1466.00 | 5.05 | 16.96 | 25 | 101.4 |
| 3 | 296.94 | 6.46 | 6.41 | 99.33 | 98.71 | 2710.00 | 9.25 | 31.03 | 40 | 101.4 |

TABLE 4
Data Simulated Using Fractionation Processes

| | | | | | | | Reboiler | | | |
|---|---|---|---|---|---|---|---|---|---|---|
| | Ethanol | | Distillate | | | | Duty | Reboiler | | |
| | Feed | Feed | Rate | Distillate | Ethanol | Reboiler | (MJ/kg | Duty (% | | Condenser |
| | Rate | Purity | (kg- | (mole % | Recovery | Duty | Ethanol | Ethanol | Theoretical | Pressure |
| Cases | (kg/h) | (mole %) | mole/h) | Ethanol) | (mole %) | (MJ/h) | Recovered) | HHV) | Trays | (kPa) |
| Vapor Feed | 230.00 | 50.00 | 5.06 | 92.49 | 93.60 | 5884.00 | 27.33 | 91.72 | 100 | 50 |
| Vapor Feed | 230.00 | 50.00 | 5.06 | 93.59 | 94.72 | 8437.00 | 38.73 | 129.96 | 150 | 10 |
| Vapor Feed | 230.00 | 50.00 | 5.08 | 93.88 | 95.38 | 8660.00 | 39.48 | 132.47 | 150 | 5 |
| Beer Preheat | 296.94 | 6.46 | | | | 958.50 | 3.23 | 10.83 | | 101.4 |
| Beer Still | 296.94 | 6.46 | 9.28 | 50.10 | 72.02 | 445.30 | 2.08 | 6.99 | 3 | 101.4 |

As shown in Tables 3 and 4, the fractionation processes were not able to overcome the azeotrope, while the processes of EXAMPLES 1-3 were able to produce ethanol with a purity greater than about 98 mole percent.

The embodiments of the present invention are not limited to the particular formulations, process steps, and materials disclosed herein as such formulations, process steps, and materials may vary somewhat. Moreover, the terminology employed herein is used for the purpose of describing exemplary embodiments only and the terminology is not intended to be limiting since the scope of the various embodiments of the present invention will be limited only by the appended claims and equivalents thereof. For example, temperature, stress, and time parameters may vary depending on the particular materials used.

Therefore, while embodiments of this disclosure have been described in detail with particular reference to exemplary embodiments, those skilled in the art will understand that variations and modifications can be effected within the scope of the disclosure as defined in the appended claims. Accordingly, the scope of the various embodiments of the present invention should not be limited to the above discussed embodiments, and should only be defined by the following claims and all equivalents.

All patents and other references cited herein are incorporated by reference as if fully set forth herein.

What is claimed is:

1. A method of recovering fuel grade ethanol, the method comprising:
producing a fermentation product comprising ethanol, water, microorganisms and a biomass material in a fermenter;
delivering at least a portion of the fermentation product to a beer still to produce a mixture comprising ethanol and water;
delivering at least a portion of the mixture comprising ethanol and water to an extractive distillation column;
delivering a solvent to the extractive distillation column;
distilling at least a portion of the ethanol from the extractive distillation column, leaving a raffinate comprising water and the solvent; and
collecting the at least the portion of the ethanol from the extractive distillation column, wherein the collected ethanol is a fuel grade ethanol.

2. The method of claim 1, further comprising recycling a raffinate from the beer still to the fermenter.

3. The method of claim 1, further comprising removing the water from the raffinate from the extractive distillation column.

4. The method of claim 3, further comprising recycling the water from the raffinate from the extractive distillation column to the fermenter.

5. The method of claim 3, further comprising drying the solvent of the raffinate from the extractive distillation column.

6. The method of claim 5, further comprising recycling the dry solvent to the extraction distillation column.

7. The method of claim 1, wherein the fuel grade ethanol is anhydrous ethanol.

8. The method of claim 1, wherein less than seven percent of a heating value of the fuel grade ethanol is expended in the method of recovering fuel grade ethanol.

9. The method of claim 1, wherein the solvent does not adversely affect the microorganisms.

10. A method of recovering fuel grade ethanol, the method comprising:
producing a fermentation product comprising ethanol, water, microorganisms and a biomass material in a fermenter;
delivering at least a portion of the fermentation product to a solvent extraction column;
delivering a solvent to the solvent extraction column;
extracting at least a portion of the ethanol from the fermentation product to produce an extract comprising ethanol, water, and solvent;
delivering at least a portion of the extract to an extractive distillation column;
delivering additional solvent to the extractive distillation column;
distilling at least a portion of the ethanol from the extractive distillation column, leaving a raffinate comprising water and the solvent; and
collecting the at least the portion of the ethanol from the extractive distillation column, wherein the collected ethanol is a fuel grade ethanol.

11. The method of claim 10, further comprising recycling a raffinate from the solvent extraction column to the fermenter.

12. The method of claim 10, further comprising removing the water from the raffinate from the extractive distillation column.

13. The method of claim 12, further comprising recycling the water from the raffinate from the extractive distillation column to the fermenter.

14. The method of claim 12, further comprising drying the solvent of the raffinate from the extractive distillation column.

15. The method of claim 14, further comprising recycling the dry solvent to the extraction distillation column and/or to the solvent extraction column.

16. The method of claim 12, further comprising recycling the solvent of the raffinate from the extractive distillation column to the solvent extraction column.

17. The method of claim 10, wherein the fuel grade ethanol is anhydrous ethanol.

18. The method of claim 10, wherein less than seven percent of a heating value of the fuel grade ethanol is expended in the method of recovering fuel grade ethanol.

19. A method of producing fuel grade ethanol, the method comprising:
producing a fermentation product comprising ethanol, water, microorganisms and a biomass material in a fermenter;
delivering at least a portion of the fermentation product to a beer still to produce a distillate mixture comprising ethanol and water while leaving a raffinate comprising water, ethanol, microorganisms and the biomass material;
recycling at least a portion of the raffinate of the beer still comprising water, ethanol, microorganisms and the biomass material into the fermenter;
delivering at least a portion of the distillate mixture comprising ethanol and water to an extractive distillation column;
delivering a solvent to the extractive distillation column;
distilling at least a portion of the ethanol from the extractive distillation column, leaving a raffinate comprising water and the solvent;
collecting the at least the portion of the ethanol from the extractive distillation column, wherein the collected ethanol is a fuel grade ethanol;
discharging the raffinate of the extractive distillation column from the extractive distillation column;
removing at least a portion of the water from the discharged raffinate of the extractive distillation column, leaving a wet solvent;
recycling at least a portion of the removed water from the discharged raffinate of the extractive distillation column into the fermenter;
delivering at least a portion of the wet solvent to a carrier gas stripper;
delivering a carrier gas to the carrier gas stripper;
discharging a dry solvent from the carrier gas stripper, leaving a wet carrier gas;
discharging at least a portion of the wet carrier gas from the carrier gas stripper;
removing water from the wet carrier gas to leave a dry carrier gas;
recycling at least a portion of the water removed from the wet carrier gas into the fermenter; and
recycling at least a portion of the dry carrier gas into the carrier gas stripper.

20. The method of claim 19, wherein the carrier gas is nitrogen, argon, helium, carbon dioxide, or Freon.
